(19) United States
(12) Patent Application Publication
(10) Pub. No.: US 2026/0345086 A1
(43) Pub. Date: Nov. 5, 2026

(54) WEARABLE EMOTION-MONITORING DEVICE TRAINED USING FMRI BRAIN SCAN DATA

(71) Applicant: Matter Neuroscience Inc., Boulder, CO (US)

(72) Inventor: Axel BOUCHON, Kleinmachnow (DE)

(73) Assignee: Matter Neuroscience Inc., Boulder, CO (US)

(21) Appl. No.: 19/488,933

(22) PCT Filed: May 31, 2024
(86) PCT No.: PCT/US2024/031840

§ 371 Date: Dec. 1, 2025

Related U.S. Application Data
(60) Provisional application No. 63/505,900, filed on Jun. 2, 2023.

(51) Int. Cl. A61B 5/00; A61B 5/0205; A61B 5/055; A61B 5/16

(52) U.S. Cl. CPC A61B 5/7267; A61B 5/0205; A61B 5/055; A61B 5/165

(57) ABSTRACT

A system for tracking human emotion in real-time comprises a wearable device comprising a plurality of sensors, one or more processors, and memory. The system measures, by the one or more sensors, a plurality of physical or chemical parameters of the wearer, wherein the plurality of physical or chemical parameters are indicative of autonomic nervous system activity of the wearer. The system processes the plurality of measured parameters using a machine-learning-trained algorithm to generate output data, wherein the output data comprises an indication of positive emotional activity of the wearer. The machine-learning-trained algorithm is trained, and the wearable sensors are configured, based on brain scan data and wearable sensor data collected from subjects during recall of positive emotional experiences.

### CROSS-REFERENCE TO RELATED APPLICATIONS

This application claims priority to and the benefit of U.S. Provisional Patent Application No. 63/505,900, filed Jun. 2, 2023, the contents of which are incorporated herein by reference in their entirety.

### FIELD

This disclosure relates generally to wearable devices, and more specifically to wearable bio-sensor emotion-monitoring devices trained using fMRI brain scan data.

### BACKGROUND

Research indicates that the human landscape for positive emotions comprises nine distinct emotion types: enthusiasm, sexual desire, recognition/pride, nurturant/family love, contentment, friendship love, amusement, pleasure, and gratitude. These emotions are created from various combinations of one or more of six reward systems: dopamine, testosterone, serotonin, oxytocin, cannabinoids, opioids. Furthermore, research has investigated the manner in which certain human emotional responses correlate to autonomic nervous system activity, for example as evidenced by cardiac interbeat interval, cardiac pre-ejection period, skin conductance responses, respiratory sinus arrhythmia, and mean arterial pressure.

### SUMMARY

Despite research into the correlation between human emotional responses and autonomic nervous system activity, real-time accurate monitoring of human emotions (including positive human emotions) using physical and chemical sensor devices to monitor autonomic nervous system activity and to determine emotional states therefrom has not yet been achieved.

Commercially available fitness trackers use sensors are able to detect body temperature, heart rate variability, blood pulse volume, oxygen saturation (SpO2) maximum, breathing rate, and/or skin conductivity (correlated to sweating). The primary focus of these fitness trackers is to identify classical fitness metrics and indicators for sleep quality.

However, available fitness trackers and other available wearables are unable to accurately track emotional responses in real time, including at least by being unable to differentiate between any different positive emotions or to determine intensity of a positive emotional experience.

Using fMRI brain scan data (3T and 7T), it has been shown that the above-mentioned nine positive emotions can be accurately and effectively identified, differentiated from one another, and quantified. However, reliance on fMRI brain scan data to recognize emotional responses limits any real-time emotion tracking to situations in which a subject is immobilized in an fMRI monitoring setting.

Thus, there is a need for improved systems and methods for real-time monitoring of human emotional responses that allows for accurate, effective, real-time differentiation between and quantification of different positive emotions. Disclosed herein are systems and methods that may address this need.

In some embodiments, a wearable device is provided comprising a plurality of sensors configured to measure physical and chemical parameters of the wearer, wherein the measured parameters are indicative of autonomic nervous system activity of the wearer. The measured parameters may be analyzed by one or more machine-learning-trained algorithms, either at the wearable device or remotely (e.g., at an associated device or server) to determine, based on the measured parameters, information about emotions of the wearer. The determined information may include an identification and quantification of an emotional state of the user and/or of an emotional reward center (e.g., a neurotransmitter) of the user. The machine-learning-trained algorithm may be trained based on labeled training data comprising (a) sensor data collected from physical and chemical sensors measuring autonomic nervous system activity of subjects and/or (b) brain scan data collected from fMRI devices from subjects. Training data may be collected while subjects experience and/or recall emotional states, and may be labeled according to subjective analysis/experience of said emotional states. The one or more sensors of the wearable device may further be configured (e.g., calibrated) based on the training data. The determined information identifying and quantifying emotions of the wearer may be stored, transmitted, used to generate one or more outputs, and/or used to trigger automatic system functionality.

In some embodiments, a system for tracking human emotion in real-time is provided, the system comprising: a wearable device comprising a plurality of sensors; one or more processors; and memory storing instructions configured to cause the system to: measure, by the one or more sensors, a plurality of physical or chemical parameters of the wearer, wherein the plurality of physical or chemical parameters are indicative of autonomic nervous system activity of the wearer; and process the plurality of measured parameters using a machine-learning-trained algorithm to generate output data, wherein the output data comprises an indication of positive emotional activity of the wearer.

In some embodiments, the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.

In some embodiments, the plurality of measured parameters comprise a parameter selected from the set comprising: cardiac interbeat interval; cardiac pre-ejection period; number of skin conductance responses; and respiratory sinus arrhythmia; and mean arterial pressure.

In some embodiments, the indication of positive emotional activity of the wearer comprises a score quantifying an emotion of the user selected from the set comprising: enthusiasm; sexual desire; recognition/pride; nurturant/family love; contentment; friendship love; amusement; pleasure; and gratitude.

In some embodiments, the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.

In some embodiments, the indication of positive emotional activity comprises a score quantifying an active emotional reward center of the user selected from the set comprising: dopamine; testosterone; serotonin; oxytocin; cannabinoids; and opioids.

In some embodiments, the instructions are configured to cause the system to provide an output to the wearer indicative of the determined positive emotional activity of the wearer, wherein the output is selected from the set comprising: a displayed visualization; an audio output; and a haptic output.

In some embodiments, the instructions are configured to cause the system to: receive training data comprising brain scan data indicating brain activity of one or more training subjects, wherein the training data is labeled with information indicating positive emotional activity of the one or more training subjects at or near the time the respective sensor data was collected; and train, using the training data, the machine-learning-trained algorithm.

In some embodiments: the training data comprising sensor data indicating a plurality of physical or chemical parameters of the training subjects, wherein the plurality of physical or chemical parameters of the one or more training subjects are indicative of autonomic nervous system activity of the training subjects; and the sensor data of the training data and the brain scan data of the training data were recorded simultaneously.

In some embodiments, the instructions are configured to cause the system to: receive training data comprising brain scan data indicating brain activity of one or more training subjects, wherein the training data is labeled with information indicating positive emotional activity of the one or more training subjects at or near the time the respective sensor data was collected; and configure, based on the training data, one or more of the plurality of sensors of the wearable device.

In some embodiments, a method for tracking human emotion in real-time is provided, the method performed at a system comprising a wearable device comprising a plurality of sensors, one or more processors, and memory, the method comprising: measuring, by the one or more sensors, a plurality of physical or chemical parameters of the wearer, wherein the plurality of physical or chemical parameters are indicative of autonomic nervous system activity of the wearer; and processing the plurality of measured parameters using a machine-learning-trained algorithm to generate output data, wherein the output data comprises an indication of positive emotional activity of the wearer.

In some embodiments, a non-transitory computer-readable storage medium storing instructions for tracking human emotion in real-time is provided, the instructions configured to be executed by one or more processors of a system comprising a wearable device comprising a plurality of sensors, to cause the system to: measure, by the one or more sensors, a plurality of physical or chemical parameters of the wearer, wherein the plurality of physical or chemical parameters are indicative of autonomic nervous system activity of the wearer; and process the plurality of measured parameters using a machine-learning-trained algorithm to generate output data, wherein the output data comprises an indication of positive emotional activity of the wearer.

In some embodiments, a wearable device for tracking human emotion in real-time is provided, the wearable device comprising: a plurality of sensors; one or more processors; and memory storing instructions configured to cause the system to: measure, by the one or more sensors, a plurality of physical or chemical parameters of the wearer, wherein the plurality of physical or chemical parameters are indicative of autonomic nervous system activity of the wearer; process the plurality of measured parameters using a machine-learning-trained algorithm to generate output data, wherein the output data comprises an indication of positive emotional activity of the wearer, wherein the machine-learning-trained algorithm is trained using training data comprising brain scan data indicating brain activity of one or more training subjects, wherein the training data is labeled with information indicating positive emotional activity of the one or more training subjects at or near the time the respective sensor data was collected; and provide an output to the wearer indicative of the determined positive emotional activity of the wearer.

In some embodiments, a method for tracking human emotion in real-time is provided, method performed by a wearable device comprising a plurality of sensors, one or more processors, and memory, the method comprising: measuring, by the one or more sensors, a plurality of physical or chemical parameters of the wearer, wherein the plurality of physical or chemical parameters are indicative of autonomic nervous system activity of the wearer; processing the plurality of measured parameters using a machine-learning-trained algorithm to generate output data, wherein the output data comprises an indication of positive emotional activity of the wearer, wherein the machine-learning-trained algorithm is trained using training data comprising brain scan data indicating brain activity of one or more training subjects, wherein the training data is labeled with information indicating positive emotional activity of the one or more training subjects at or near the time the respective sensor data was collected; and providing an output to the wearer indicative of the determined positive emotional activity of the wearer.

In some embodiments, a non-transitory computer-readable storage medium storing instructions for tracking human emotion in real-time, the instructions configured to be executed by one or more processors of a wearable device comprising a plurality of sensors, to cause the wearable device to: measure, by the one or more sensors, a plurality of physical or chemical parameters of the wearer, wherein the plurality of physical or chemical parameters are indicative of autonomic nervous system activity of the wearer; process the plurality of measured parameters using a machine-learning-trained algorithm to generate output data, wherein the output data comprises an indication of positive emotional activity of the wearer, wherein the machine-learning-trained algorithm is trained using training data comprising brain scan data indicating brain activity of one or more training subjects, wherein the training data is labeled with information indicating positive emotional activity of the one or more training subjects at or near the time the respective sensor data was collected; and provide an output to the wearer indicative of the determined positive emotional activity of the wearer.

Any of the embodiments disclosed herein may be combined, in whole or in part, with any other embodiment disclosed herein.

### BRIEF DESCRIPTION OF THE FIGURES

FIG. 1 shows an exemplary system for tracking human emotions in real time using a wearable device, according to some embodiments.

FIG. 2 shows an exemplary positive emotion to neurotransmitter (PE-NT) matrix, according to some embodiments.

FIG. 3 shows an exemplary method for tracking human emotions in real time using a wearable device, according to some embodiments.

FIG. 4 shows an exemplary computer, according to some embodiments.

### DETAILED DESCRIPTION

In the following description of the disclosure and embodiments, reference is made to the accompanying drawings in which are shown, by way of illustration, specific embodiments that can be practiced. It is to be understood that other embodiments and examples can be practiced, and changes can be made, without departing from the scope of the disclosure.

In addition, it is also to be understood that the singular forms “a,” “an,” and “the” used in the following description are intended to include the plural forms as well unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Unless specifically stated otherwise as apparent from the following discussion, it is appreciated that, throughout the description, discussions utilizing terms such as “processing,” “computing,” “calculating,” “determining,” “displaying,” or the like refer to the action and processes of a computer system, or similar electronic computing device, that manipulates and transforms data represented as physical (electronic) quantities within the computer system memories or registers or other such information storage, transmission, or display devices.

Certain aspects of the present disclosure include process steps and instructions described herein in the form of an algorithm. It should be noted that the process steps and instructions of the present disclosure could be embodied in software, firmware, or hardware, and, when embodied in software, they could be downloaded to reside on and be operated from different platforms used by a variety of operating systems.

The term “machine learning” as used throughout the present disclosure is understood to include artificial intelligence (AI) systems that learn to perform tasks based on training data, including machine learning-based generative AI, neural networks (e.g., convolutional neural networks (CNN), recurrent neural networks (RNN), etc.), generative adversarial networks (GANs), deep learning, computer vision, and/or other variations thereof. A model trained using machine learning training processes may be referred to herein as a machine-learning-trained model or a machine-learning-trained algorithm.

The present disclosure also relates to a device for performing the operations herein. This device may be specially constructed for the required purposes or it may comprise a general-purpose computer selectively activated or reconfigured by a computer program stored in the computer. Such a computer program may be stored in a non-transitory, computer-readable storage medium such as, but not limited to, any type of disk, including floppy disks, optical disks, CD-ROMs, magnetic-optical disks, read-only memories (ROMs), random access memories (RAMs), EPROMs, EEPROMs, magnetic or optical cards, application-specific integrated circuits (ASICs), or any type of media suitable for storing electronic instructions and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present disclosure is not described with reference to any particular programming language. It will be appreciated that a variety of programming languages may be used to implement the teachings of the present disclosure as described herein.

As used herein, the term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 1 millisecond (ms), 5 ms, 0.01 seconds, 0.05 seconds, 0.1 seconds, 0.5 seconds, 1 second, 0.1 minute, 0.5 minutes, 1 minute, or more. In some cases, a real time event may be performed almost immediately or within a short enough time span, such as within at most 1 second, 0.5 seconds, 0.1 seconds, 0.05 seconds, 0.01 seconds, 5 ms, 1 ms, or less.

Described herein are systems and methods that enable real-time monitoring of human emotional responses that allows for accurate, effective, real-time differentiation between and quantification of different positive emotions.

### Systems & Devices

In some embodiments, a system 100 for monitoring human emotional responses is provided. System 100 may include wearable device 102 and, optionally, remote system 110. Wearable device 102 may include a plurality of physical and/or chemical sensors 104 for monitoring biological responses indicative of autonomic nervous system activity of the wearer. For example, the wearable device may include one or more sensors to measure one or more parameters including, but not limited to:

Cardiac Interbeat Interval (CBI or IBI) (ms), as measured, for example, by sensors based on (1) electrical activity (such as Electrocardiogram based on wet electrodes, dry electrodes, or capacitive electrodes), (2) sensors detecting arterial pulse using photoplethysmography (PPG), or sensors such as PhysioCam (PhyC), a non-contact system capable of measuring arterial pulse with sufficient precision to derive HRV during different challenges, (3) sensors based on mechanical activity (balistocardiogram (BCG) using e.g.

Hydraulic sensors, EMFi film sensors, Accelerometer), radio frequency or seismocardiogram (SCG) using e.g. Accelerometer, Laser Doppler Vibrometer, Laser Speckle Vibrometry, Airborne Ultrasound or gyrocardiogram (GCG) using gyroscope or Laser speckle vibrometer, and/or (4) Forcecardiography;

Cardiac Pre-Ejection Period (PEP) (ms), as measured, for example, by one or more of the same or similar sensor types as described above with reference to IBI (optionally, with a preference for Forcecardiography and/or Seismocardiography). PEP may be measured by simultaneously collecting both ECG, as described earlier, and impedance cardiography;

Number of valid Skin Conductance Responses (SCRs), as measured, for example, by sensors detecting galvanic skin response such as Ag/AgCl, stainless steel, silver, brass, and gold electrodes, Flexcomp Infiniti physiological monitoring and data acquisition unit, Empatica E4 and Refa System, Microsoft Band 2, Empatica E4, Health Sensor Platform, BITalino, Polar H6, Wearable Zephyr BioHarness 3, and/or Obimon EDA;

Respiratory Sinus Arrhythmia (RSA) (ms2), as measured, for example, by an electrocardiogram sensors such as any one or more of those described above; and/or Mean Arterial Pressure (MAP) (mmHg), as measured, for example, by (1) pressure-based methods (e.g., vascular unloading technique, arterial tonometry), (2) ultrasound-based methods, and/or (3) deep-learning based methods using data from PPG or ECG.

Measured parameters may be stored in local data storage 107 provided as a part of device 102 for local analysis by one or more processors 106 provided as a part of device 102. Additionally or alternatively, measured parameters may be transmitted via network communication device 108 to remote system 110, for example for remote storage, remote display, and/or remote data processing.

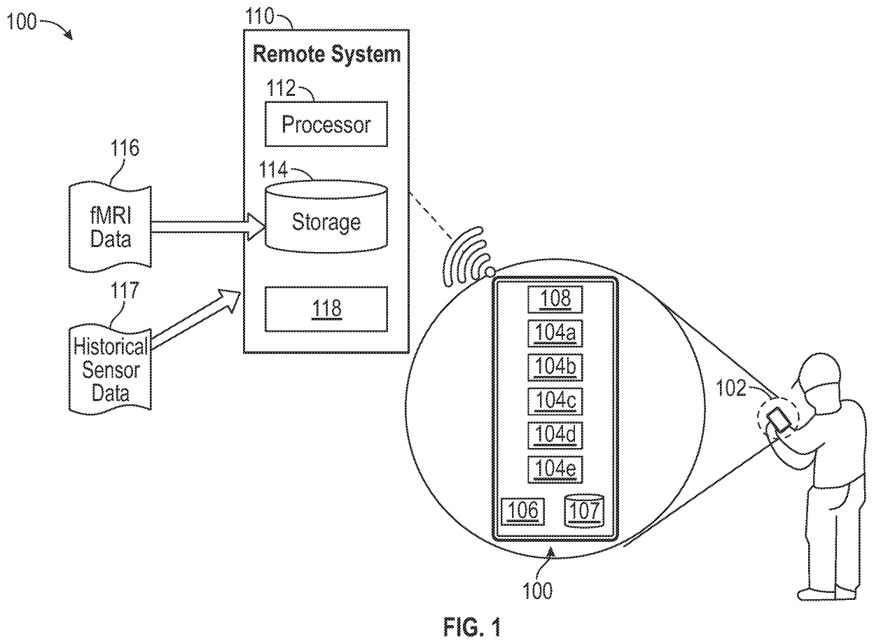

As shown in FIG. 1, remote system 110 may include network communication device 118 configured to receive data from and/or send data to one or more wearable devices such as wearable device 102. In the example of system 100, remote system 100 may receive the measured parameters transmitted from wearable device 102 and may store the parameters in storage 114. Optionally, one or more processors 112 of remote system 110 may process the received parameters.

In some embodiments, the one or more measured parameters may be processed—whether locally at wearable device 102 by one or more processors 106 and/or remotely at remote system 110 by one or more processors 112—to determine an emotional state of the wearer of wearable device 102, and/or to determine emotional reward systems active in the wearer, based on the measured parameters. The emotional reward systems may include one or more of dopamine, testosterone, serotonin, oxytocin, cannabinoids, and opioids.

Processing may take place in real-time or near-real-time. One or more of the measured parameters may be processed, for example using one or more algorithms, rules, and/or trained models to identify the presence of one or more emotional responses (and/or one or more emotional reward systems) and to quantify one or more of the identified emotional responses (and/or reward systems). In some embodiments, any one or more of the nine positive human emotional responses-enthusiasm, sexual desire, recognition/pride, nurturant/family love, contentment, friendship love, amusement, pleasure, and gratitude-may be identified, and a respective quantification for any one or more of the identified human emotional responses may be generated. In some embodiments, a score quantifying an intensity of an identified specific emotional response may be generated. In some embodiments, any one or more of the emotional reward centers indicated above may be identified, and a respective quantification for any one or more of the identified emotional reward centers may be generated. In some embodiments, a score quantifying an intensity of an identified specific emotional reward center may be generated.

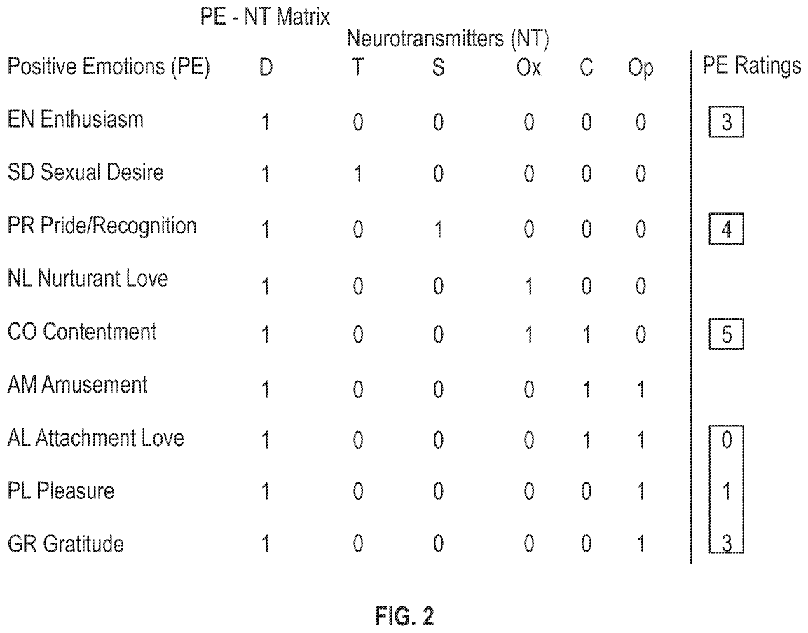

In some embodiments, the system may be configured to apply one or more algorithms (e.g., including one or more algorithms applied along with and/or as part of the machine-learning-trained algorithms described herein) to identify and/or quantify emotional responses and/or emotional reward systems, mentioned above, and to translate them into neurotransmitters (and associated neurotransmitter levels). For example, said algorithms can use a positive emotion to neurotransmitter (PE-NT) matrix, an example of which is illustrated in FIG. 2, that can translate a quantified positive emotion into an amount of one or more associated neurotransmitters for different emotional reward systems. An exemplary PE-NT matrix is described, for example, in U.S. patent application Ser. No. 17/389,023, which is hereby incorporated by reference in its entirety.

The rows of the PE-NT matrix can represent the positive emotion categories such as enthusiasm, sexual desire, pride/recognition, nurturant love, contentment, amusement, pleasure, and gratitude, which correspond to the emotional states input by a user and/or identified by the system. The columns of the PE-NT matrix can represent the neurotransmitters (dopamine, testosterone, Serotonin, oxytocin, cannabinoids, and opioids) associated with the positive emotions. A “1” in the matrix can indicate that a particular neurotransmitter is associated with that particular emotion. A “0” in the matrix can indicate that a particular is not associated with that particular emotion.

The PE-NT matrix can be used to calculate the amount of a neurotransmitter associated with a media content object. In one or more examples, the calculation can include a positive emotions (“PE”) ratings column that shows the emotional state(s) provided by the user with respect to a particular media content object. Optionally, the calculation may also include an indicator of the intensity of the emotional state experienced by the user. For instance, in one example, the user may have indicated that his/her enthusiasm while consuming particular media content is mild (indicating that it is lower than average but still present) and that PE rating can be quantified as a 3. In the same example, in the PE ratings column the user may rate their contentment while consuming the media content as a 5, which is average (e.g., on a scale of 0-10). In order to calculate the amount of neurotransmitter activity associated with the media content object, the calculation can multiply the PE rating by the numbers in the PE-NT matrix to generate a number associated with the activity of a particular neurotransmitter. For instance, as shown in PE-NT matrix, enthusiasm can be associated with the release of dopamine. The PE rating for enthusiasm (provided by the user as associated with particular media content) is 3. That value is multiplied by 1 under the dopamine column to arrive at a value of 3. Thus, with respect to the enthusiasm felt by the user as expressed in the PE rating, the dopamine level associated with that enthusiasm for the media content is quantified at 3. In one or more examples, the remaining columns are left at 0 because those neurotransmitters are not associated with enthusiasm.

In some examples, the identified emotional states may correspond to the activity of two or more neurotransmitters, and the PE-NT matrix values of each of the neurotransmitters can be multiplied by the PE rating to arrive at a value for each neurotransmitter. For instance, if contentment was rated a 5 by the user and is associated with dopamine, oxytocin, and cannabinoids, each of those neurotransmitters can be multiplied by 5 (multiplying 5×1) to determine the level of neurotransmitter activity corresponding to that positive emotion. Once the calculation is made for each of the emotional states experienced by the user for each neurotransmitter, the calculation can add up the totals for each neurotransmitter and associate the neurotransmitter totals with the media content object.

In some embodiments, processing the one or more measured parameters to determine (e.g., identify and/or quantify) an emotional response and/or a reward system may be performed by one or more algorithms that have been trained with machine learning using functional magnetic resonance imaging (fMRI) brain scan data and using data from one or more wearable sensors such as the sensors 104 included in wearable device 102. In some embodiments, in addition to or instead of utilizing ML training procedures to train based on brain scan data, one or more deterministic algorithms or other calculations may process the brain scan data and/or the wearable sensor data to determine correlations between the emotional response and/or reward system, to build deterministic models about relationships between wearable sensor data and emotional responses and/or reward systems, and/or to deterministically process and analyze wearable sensor data to determine and quantify emotional responses and/or reward systems.

As shown in FIG. 1, storage 114 of remote system 110 may store fMRI data 116 and/or historical sensor data 117. Historical sensor data 117 may be received from sensors 104 of wearable device 102 and/or from one or more similar sensors. fMRI data 116 may be received from one or more MRI devices. In some examples, historical sensor data 117 may include data received from sensors 104 of wearable device 102, wherein said data is currently and/or was previously stored in local data storage 107 of device 102, and has been transmitted from device 102 to remote system 110. In some embodiments, fMRI data 116 and historical sensor data 117 may be acquired at the same time from the same subject, such that certain data points from the two different data sources correspond to one another. In some embodiments, fMRI data 116 and historical sensor data 117 may be acquired at different times. Historical sensor data 117 and/or fMRI data 116 may be acquired for a single subject or for a plurality of subjects. Some or all of historical sensor data 117 and/or fMRI data 116 may be unlabeled and/or may be labeled, for example with one or more labels indicating a subject to whom the data corresponds, a time of collection, a sensor used for collection, other associated (e.g., simultaneous) data points, and/or one or more associated memories or emotions that were being experienced or recalled during the time at which (or shortly before) the data was collected.

In some embodiments, historical sensor data 117 and/or fMRI data 116 may be used as training data to train, e.g., using machine learning, one or more algorithms configured to identify and quantify human emotional responses (and/or emotional reward centers) based on parameters indicative of autonomic nervous system responses, as measured by sensors (such as sensors 104). The one or more sensors used to measure historical sensor data 117 may be configured to be operable within an operating MRI machine, such that brain scan (fMRI) training data and wearable sensor training data may be simultaneously collected. In some embodiments, wearable device 102 may similarly be configured to be operable within an operating MRI machine. In some embodiments, the one or more algorithms may be trained using machine learning based in part on fMRI data, but, in use, may not require (or necessarily accept) fMRI data as input once deployed. Instead, the one or more machine-learning-trained algorithms may be trained such that, in practice, they determine and quantify human emotional responses (and/or emotional reward centers) based on parameters indicative of autonomic nervous system responses alone.

Gathering the fMRI data 116 and the historical sensor data 117 can occur as follows. Upon memory recall, a brain scanner, e.g., 3 Tesla (T) fMRI or 7 Tesla (T) fMRI, can be used to determine a 3D location of brain activity as well as to detect the brain activation intensity in said location of the brain. One or more of the nine positive emotions (listed above) may be identified based on the distinct, characteristic brain areas/nuclei in which activity is detected based on the fMRI data. The brain activation intensity in a distinct nucleus can correspond to the emotion intensity subjectively experienced by a person. These two data sets gathered from fMRI-3D location of brain activity identifying a distinct positive emotion and the respective brain activity identifying a respective emotion intensity-can be correlated with single or multiple peripheral data (e.g., autonomic nervous system responses) recorded by the various wearable sensors while being in the brain scanner (recorded as the historical sensor data 117). In some examples, the wearable sensors may measure skin conductivity, breathing, and heart data.

In prior systems, low signal-to-noise ratio of wearable sensor data has made it impossible to identify distinct signals of positive emotions based solely on the wearable sensor data. However, as described herein, using the highly sensitive fMRI data in real-time to first interpret and then optimize wearable sensor data allows a significant increase of signal-to-noise ratio. Based on this correlation, in some examples, real-life experiences triggering positive emotions can be recorded by the wearable device and then later can be reassessed in the fMRI (artificially reducing noise) for further optimization of the signal-to-noise ratio. Given the highly universal nature of emotions among all humans, a wearable device trained by fMRI data of 20-30 testers can be used broadly and does not need an individual training by personal brain (e.g., fMRI) and wearable sensor data.

In some embodiments, the training data (e.g., the fMRI data 116 and/or the historical sensor data 117) may be gathered while the subject is in a still position, such as when the subject is lying still within a brain scanner. This can prevent other electrophysiological signals, such as those related to muscle movement, from producing noise that impairs the ability to gather the data.

In some embodiments, the algorithm(s) trained to identify and quantify emotional responses and/or emotional rewards systems based on the fMRI data and wearable sensor data may be stored at remote system 110 (e.g., on storage 114), at local data storage 107 on wearable device 102, elsewhere in system 100, or at a system separate from system 100.

In some embodiments, one or more of sensors 104 of wearable device 102 may be optimized for sensitivity and/or accuracy based on fMRI brain data and/or personal subjective data. For example, system 100 may simultaneously collect sensor data from sensors 104, fMRI brain scan data (e.g., using a brain scanner) from the same subject, as well as personal subjective data from the subject. The personal subjective data may include audio data, textual data, etc. describing the emotional aspects of the subject's experience or memory. Based on this collected data, system 100 may calibrate, configure, and/or otherwise optimize the sensitivity and/or accuracy of sensors 104.

In some embodiments, one or more of sensors 104 may be configured (e.g., calibrated, optimized), and/or the algorithm may be trained using machine learning, such that the sensors 104 and/or machine-learning-trained algorithm may be deployed for use by the general population without personalized training or personalized calibration required. In some embodiments, personalized training and/or personalized calibration may be used to optimize performance for a specific user. Personalized training and/or calibration may include any one or more aspects of the above-described training and calibration process described for a general population.

After the algorithm has been trained and/or the sensors have been configured (e.g., calibrated, optimized), the machine-learning-trained algorithm may be applied (e.g., deployed) to process new (e.g., unlabeled) sensor data collected from sensors 104 of wearable device 102. The new sensor data may be unlabeled, and therefore may not explicitly indicate any association with a memory, experience, human emotion, or emotional reward center. By processing the unlabeled sensor data using the machine-learning-trained algorithm, the system may generate output data that includes (a) an indication of one or more human emotions (e.g., any one or more of the positive human emotions described above) and a quantification for any one or more of the identified human emotions and/or (b) an indication of one or more emotional reward systems (e.g., any one or more of the emotional reward systems described above) and a quantification for any one or more of the identified emotional reward systems. The output data may include a score indicating the quantification of a given human emotion or reward system and/or a confidence level associated with the determination of the given human emotion and/or reward system.

The output score may be stored locally and/or remotely (e.g., on storage 107 and/or on storage 114), transmitted to one or more other system components and/or to one or more other systems, may be used to generate one or more visualizations and/or one or more other outputs (e.g., auditory outputs and/or haptic feedback), and/or may be used to trigger one or more automated system functionalities.

As noted above, the training data (e.g., the fMRI data and/or the wearable sensor data) may be collected while the subject is in a still position. However, when the device is deployed, sensor data (e.g., autonomic nervous system data) can be collected at any time, including when the wearer is moving. Thus, other electrophysiological signals produced by the wearer, such as those related to muscle movement, can affect the ability of the wearable sensors to effectively collect data (due to the low signal-to-noise ratio of the autonomic nervous system data). Training data gathered using the methods described above may not be collectable for training the algorithm(s) using machine learning to account for this variation in data, because it may not be possible to obtain fMRI data while the subject is moving.

Despite this problem, the systems and devices herein can be configured to efficaciously identify and quantify emotional responses and/or reward systems based on wearable sensor data gathered at any time, including during movement of the wearer, and analyzed using algorithms trained using machine learning and at least in part on brain scan (e.g., fMRI) data. For example, the systems and devices described herein may be configured to account for electrophysiological signals using a “stepwise” training regimen of the algorithm(s). The training regimen can include multiple stages that progressively introduce movement of the wearer so that, in use, the machine-learning-trained algorithm(s) configured to identify and quantify emotional responses and/or rewards systems can do so despite movement of the wearer. During each stage of the training, the same positive memory triggers may be presented to the subject.

In some examples, a first stage of training can include collecting wearable sensor training data in conjunction with brain scan training data, as described above. During this stage of training, the subject may be in a still position in an fMRI brain scanner. The first stage of training can include a post-hoc emotion assessment in which the wearer provides subjective ratings of his/her emotions, which can be used to calibrate and/or optimize the wearable device.

In some examples, a second stage of machine-learning-training the algorithms can include collecting additional wearable sensor data while the subject is in a still, standing position outside of the fMRI brain scan environment. Any noise that is detected from basic muscle contraction due to the subject standing can be measured and removed from the measured wearable data. In some examples, during the second stage of training the subject can again complete the post-hoc emotion assessment to subjectively rate his/her emotions.

In some examples, a third stage of machine-learning-training the algorithms can include collecting additional wearable sensor training data while the subject is walking on a treadmill. Any noise that is detected due to active muscle contraction while walking can be measured and removed from the measured wearable data. In some examples, during the second stage of training the subject can again complete the post-hoc emotion assessment to subjectively rate his/her emotions.

In some examples, following the three stages of training described above, the wearable device may be further trained using a continuous guided machine-learning training process. This can include the sensors of the wearable device ad-hoc detecting signals above a noise baseline, and, based on the detection, prompting the wearer of the device via an input device for confirmation of the detected event. Based on the subject's input, e.g., confirming the detected event, the input device may prompt the subject for additional information on the event, such as for an indication of the type of emotion experienced. In some examples, the input device may prompt the user for a rating that quantifies the intensity of the emotion experienced. Based on the subject's input, the machine-learning-trained algorithm(s) configured to identify and quantify emotional responses and/or reward centers may be refined. In some examples, the wearable device may be configured to detect signals indicative of a positive event. In the instance a negative event is detected, the system may use the user's input indicating that negative event was detected to further refine the machine-learning-trained algorithm(s). The continuous ML training process may be conducted for a period of time, such as about 2-3 weeks.

### Methods

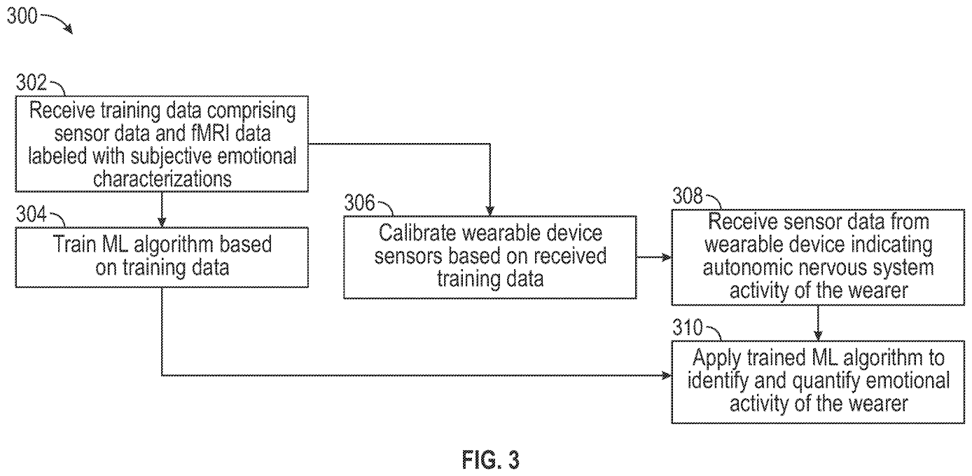

FIG. 3 shows an exemplary method 300 for tracking human emotional activity using a wearable device. Method 300 may be performed by a system for tracking human emotional activity, such as system 100 as described herein.

At block 302, the system may receive training data. As described herein, the training data may comprise (a) sensor data from sensors that measure physical and/or chemical activity indicative of autonomic nervous system activity and/or (b) brain scan (e.g., fMRI) data. The training data may be labeled to indicate information regarding emotional activity (e.g., subjectively reported emotional states and/or neurotransmitter levels) of the subject at the time the training data was measured. In some embodiments, an initial round of training data may be collected while a subject is wearing a wearable sensor and in an fMRI machine, such that wearable sensor data and brain scan data can be simultaneously collected. The data (sensor data and brain scan data) can be collected while the subject recalls positive memories that have been subjectively characterized as associated with one or more positive emotions and emotion intensities, respectively. The subjective emotional characterizations by the subject may be used to label the training data that is collected.

At block 304, the system may train an algorithm using machine learning based on the received training data. As described herein, the machine-learning-trained algorithm may be trained based on the labeled sensor training data and/or based on the labeled brain scan training data such that the machine-learning-trained algorithm is configured to accept unlabeled wearable sensor data (indicative of autonomic nervous system activity of the wearer) and to generate output data identifying and quantifying emotional activity of the user. The identified and quantified emotional activity may include identification and quantification of one or more emotional states, as described above, and/or of one or more emotional reward centers (e.g., neurotransmitters), as described above.

At block 306, the system may configure the wearable sensors based on the received training data. As described herein, one or more sensors of a wearable device may be calibrated, optimized, and/or otherwise configured based on the labeled training data. In instances in which brain scan data and sensor data (e.g., autonomic nervous system data) are collected simultaneously, the wearable sensors may be calibrated based on relationships between the brain scan data and the sensor data (e.g., autonomic nervous system data).

Calibration may utilize a peak memory for a given emotional state (e.g., a memory having a maximum intensity score of, for example, 8, and maximum brain activation in the region of interest) as well as a control memory that is rated at 0 for the given emotional state (e.g., a memory having an intensity score of 0 and minimum or baseline brain activation in the region of interest).

At block 308, the system may receive sensor data from the configured wearable sensors. As described herein, the calibrated and deployed wearable device may measure sensor data (e.g., using the same or similar sensor types as were used in collecting the training data) indicative of autonomic nervous system activity of the wearer. The collected data from the wearable device may, in some embodiments, not be collected along with any corresponding brain scan data and may, in some embodiments, not be labeled with any explicit indication of associated emotional activity.

At block 310, the system may apply the machine-learning-trained algorithm to process the received sensor data to identify and quantify emotional activity of the wearer of the wearable sensors. The machine-learning-trained algorithm may generate output data comprising an identification and quantification of one or more emotional states as described above and/or of one or more emotional reward centers (e.g., neurotransmitters) as described above. The generated output data may be stored, transmitted, used to generate one or more outputs (e.g., at the wearable device), and/or used to trigger one or more automatic system functionalities.

The method described above with reference to blocks 302-310 may be validated, repeated, and/or iterated one or more times. For example, after the wearable sensor has been configured and deployed and used at blocks 308-310 to identify and quantify positive emotional activity of the wearer, the experiences associated with said positive emotional activity may be noted for later recall by the subject during subsequent fMRI training data recording sessions. During subsequent fMRI training data recording sessions, the subject may recall positive memories associated with the experience for which the wearable sensor previously identified and quantified the real-time positive emotional activity of the subject.

During recall, brain scan data may be collected and, optionally, additional wearable sensor data may be collected. The system may compare the wearable sensor data from the actual experience to the wearable sensor data during recall of said experience in order to further calibrate and/or validate the sensor configurations. Furthermore, the system may use the newly recorded brain scan data, the newly recorded wearable sensor data during recall, and/or the real-time sensor data recorded during the actual experience to update the machine-learning-trained algorithm (e.g., to re-train the algorithm) and/or to update calibration or other configuration of one or more of the sensors of the wearable device.

### Computing Device

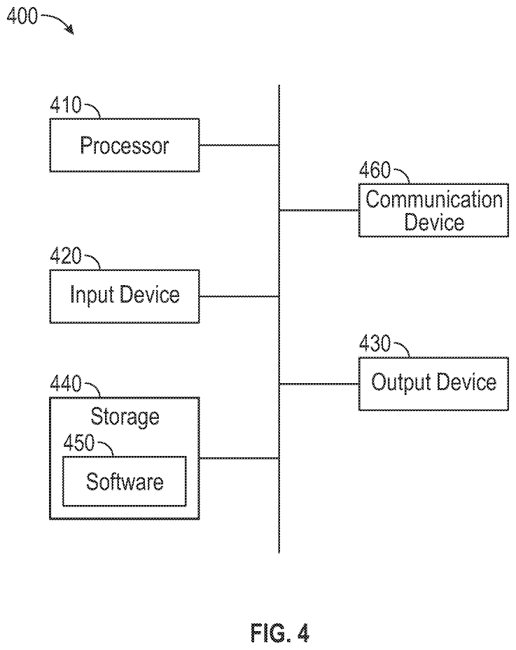

FIG. 4 illustrates an example of a computer, in accordance with some embodiments. Device 400 can be a host computer connected to a network. Device 400 can be a client computer or a server. As shown in FIG. 4, device 400 can be any suitable type of microprocessor-based device, such as a personal computer, workstation, server, or handheld computing device (portable electronic device) such as a phone or tablet. The device can include, for example, one or more of processor 410, input device 420, output device 430, storage 440, and communication device 460. Input device 420 and output device 430 can generally correspond to those described above and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage, such as an electrical, magnetic, or optical memory, including a RAM, cache, hard drive, or removable storage disk. Communication device 460 can include any suitable device capable of transmitting and receiving signals over a network, such as a network interface chip or device. The components of the computer can be connected in any suitable manner, such as via a physical bus or wirelessly.

Software 450, which can be stored in storage 440 and executed by processor 410, can include, for example, the programming that embodies the functionality of the present disclosure (e.g., as embodied in the devices as described above).

Software 450 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 440, that can contain or store programming for use by or in connection with an instruction execution system, apparatus, or device.

Software 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can implement any operating system suitable for operating on the network. Software 450 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### EXEMPLARY EMBODIMENTS

The following embodiments are exemplary and are not intended to limit the scope of any invention described herein.
Embodiment 1. A system for tracking human emotion in real-time, the system comprising:
a wearable device comprising a plurality of sensors; one or more processors; and
memory storing instructions configured to cause the system to:
   measure, by the one or more sensors, a plurality of physical or chemical parameters of the wearer, wherein the plurality of physical or chemical parameters are indicative of autonomic nervous system activity of the wearer; and
   process the plurality of measured parameters using a machine-learning-trained algorithm to generate output data, wherein the output data comprises an indication of positive emotional activity of the wearer.
Embodiment 2. The system of embodiment 1, wherein the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.
Embodiment 3. The system of any one of embodiments 1-2, wherein the plurality of measured parameters comprise a parameter selected from the set comprising: cardiac interbeat interval; cardiac pre-ejection period; number of skin conductance responses; and respiratory sinus arrhythmia; and mean arterial pressure.
Embodiment 4. The system of any one of embodiments 1-3, wherein the indication of positive emotional activity of the wearer comprises a score quantifying an emotion of the user selected from the set comprising: enthusiasm; sexual desire; recognition/pride; nurturant/family love; contentment; friendship love; amusement; pleasure; and gratitude.
Embodiment 5. The system of any one of embodiments 1-4, wherein the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.
Embodiment 6. The system of any one of embodiments 1-5, wherein the indication of positive emotional activity comprises a score quantifying an active emotional reward center of the user selected from the set comprising: dopamine; testosterone; serotonin; oxytocin; cannabinoids; and opioids.
Embodiment 7. The system of any one of embodiments 1-6, wherein the instructions are configured to cause the system to provide an output to the wearer indicative of the determined positive emotional activity of the wearer, wherein the output is selected from the set comprising: a displayed visualization; an audio output; and a haptic output.
Embodiment 8. The system of any one of embodiments 1-7, wherein the instructions are configured to cause the system to:
receive training data comprising brain scan data indicating brain activity of one or more training subjects, wherein the training data is labeled with information indicating positive emotional activity of the one or more training subjects at or near the time the respective sensor data was collected; and
train, using the training data, the machine-learning-trained algorithm.
Embodiment 9. The system of embodiment 8, wherein:
the training data comprises sensor data indicating a plurality of physical or chemical parameters of the training subjects, wherein the plurality of physical or chemical parameters of the one or more training subjects are indicative of autonomic nervous system activity of the training subjects; and
the sensor data of the training data and the brain scan data of the training data were recorded simultaneously.
Embodiment 10. The system of any one of embodiments 1-9, wherein the instructions are configured to cause the system to:
receive training data comprising brain scan data indicating brain activity of one or more training subjects, wherein the training data is labeled with information indicating positive emotional activity of the one or more training subjects at or near the time the respective sensor data was collected; and
configure, based on the training data, one or more of the plurality of sensors of the wearable device.
Embodiment 11. A method for tracking human emotion in real-time, the method performed at a system comprising a wearable device comprising a plurality of sensors, one or more processors, and memory, the method comprising:
measuring, by the one or more sensors, a plurality of physical or chemical parameters of the wearer, wherein the plurality of physical or chemical parameters are indicative of autonomic nervous system activity of the wearer; and
processing the plurality of measured parameters using a machine-learning-trained algorithm to generate output data, wherein the output data comprises an indication of positive emotional activity of the wearer.
Embodiment 12. The method of embodiment 11, wherein the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.
Embodiment 13. The method of any one of embodiments 11-12, wherein the plurality of measured parameters comprise a parameter selected from the set comprising: cardiac interbeat interval; cardiac pre-ejection period; number of skin conductance responses; and respiratory sinus arrhythmia; and mean arterial pressure.
Embodiment 14. The method of any one of embodiments 11-13, wherein the indication of positive emotional activity of the wearer comprises a score quantifying an emotion of the user selected from the set comprising: enthusiasm; sexual desire; recognition/pride; nurturant/family love; contentment; friendship love; amusement; pleasure; and gratitude.
Embodiment 15. The method of any one of embodiments 11-14, wherein the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.
Embodiment 16. The method of any one of embodiments 11-15, wherein the indication of positive emotional activity comprises a score quantifying an active emotional reward center of the user selected from the set comprising: dopamine; testosterone; serotonin; oxytocin; cannabinoids; and opioids.
Embodiment 17. The method of any one of embodiments 11-16,comprising providing an output to the wearer indicative of the determined positive emotional activity of the wearer, wherein the output is selected from the set comprising: a displayed visualization; an audio output; and a haptic output.
Embodiment 18. The method of any one of embodiments 11-17,comprising:

Receiving training data comprising brain scan data indicating brain activity of one or more training subjects, wherein the training data is labeled with information indicating positive emotional activity of the one or more training subjects at or near the time the respective sensor data was collected; and
training, using the training data, the machine-learning-trained algorithm.
Embodiment 19. The method of embodiment 18, wherein:
the training data comprises sensor data indicating a plurality of physical or chemical parameters of the training subjects, wherein the plurality of physical or chemical parameters of the one or more training subjects are indicative of autonomic nervous system activity of the training subjects; and
the sensor data of the training data and the brain scan data of the training data were recorded simultaneously.
Embodiment 20. The method of any one of embodiments 11-19,comprising:
receiving training data comprising brain scan data indicating brain activity of one or more training subjects, wherein the training data is labeled with information indicating positive emotional activity of the one or more training subjects at or near the time the respective sensor data was collected; and
configuring, based on the training data, one or more of the plurality of sensors of the wearable device.
Embodiment 21. A non-transitory computer-readable storage medium storing instructions for tracking human emotion in real-time, the instructions configured to be executed by one or more processors of a system comprising a wearable device comprising a plurality of sensors, to cause the system to:
measure, by the one or more sensors, a plurality of physical or chemical parameters of the wearer, wherein the plurality of physical or chemical parameters are indicative of autonomic nervous system activity of the wearer; and
process the plurality of measured parameters using a machine-learning-trained algorithm to generate output data, wherein the output data comprises an indication of positive emotional activity of the wearer.
Embodiment 22. The non-transitory computer-readable storage medium of embodiment 21, wherein the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.
Embodiment 23. The non-transitory computer-readable storage medium of any one of embodiments 21-22, wherein the plurality of measured parameters comprise a parameter selected from the set comprising: cardiac interbeat interval; cardiac pre-ejection period; number of skin conductance responses; and respiratory sinus arrhythmia; and mean arterial pressure.
Embodiment 24. The non-transitory computer-readable storage medium of any one of embodiments 21-23, wherein the indication of positive emotional activity of the wearer comprises a score quantifying an emotion of the user selected from the set comprising: enthusiasm; sexual desire; recognition/pride; nurturant/family love; contentment; friendship love; amusement; pleasure; and gratitude.
Embodiment 25. The non-transitory computer-readable storage medium of any one of embodiments 21-24, wherein the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.
Embodiment 26. The non-transitory computer-readable storage medium of any one of embodiments 21-25, wherein the indication of positive emotional activity comprises a score quantifying an active emotional reward center of the user selected from the set comprising: dopamine; testosterone; serotonin; oxytocin; cannabinoids; and opioids.
Embodiment 27. The non-transitory computer-readable storage medium of any one of embodiments 21-26, wherein the instructions are configured to cause the system to provide an output to the wearer indicative of the determined positive emotional activity of the wearer, wherein the output is selected from the set comprising: a displayed visualization; an audio output; and a haptic output.
Embodiment 28. The non-transitory computer-readable storage medium of any one of embodiments 21-27, wherein the instructions are configured to cause the system to:
receive training data comprising brain scan data indicating brain activity of one or more training subjects, wherein the training data is labeled with information indicating positive emotional activity of the one or more training subjects at or near the time the respective sensor data was collected; and
train, using the training data, the machine-learning-trained algorithm.
Embodiment 29. The non-transitory computer-readable storage medium of embodiment 28, wherein:
the training data comprises sensor data indicating a plurality of physical or chemical parameters of the training subjects, wherein the plurality of physical or chemical parameters of the one or more training subjects are indicative of autonomic nervous system activity of the training subjects; and
the sensor data of the training data and the brain scan data of the training data were recorded simultaneously.
Embodiment 30. The non-transitory computer-readable storage medium of any one of embodiments 21-29, wherein the instructions are configured to cause the system to:
receive training data comprising brain scan data indicating brain activity of one or more training subjects, wherein the training data is labeled with information indicating positive emotional activity of the one or more training subjects at or near the time the respective sensor data was collected; and
configure, based on the training data, one or more of the plurality of sensors of the wearable device.
Embodiment 31. A wearable device for tracking human emotion in real-time, the wearable device comprising:
a plurality of sensors;
one or more processors; and
memory storing instructions configured to cause the device to:
   measure, by the one or more sensors, a plurality of physical or chemical parameters of the wearer, wherein the plurality of physical or chemical parameters are indicative of autonomic nervous system activity of the wearer;
   process the plurality of measured parameters using a machine-learning-trained algorithm to generate output data, wherein the output data comprises an indication of positive emotional activity of the wearer, wherein the machine-learning-trained algorithm is trained using training data comprising brain scan data indicating brain activity of one or more training subjects, wherein the training data is labeled with information indicating positive emotional activity of the one or more training subjects at or near the time the respective sensor data was collected; and
   provide an output to the wearer indicative of the determined positive emotional activity of the wearer.
Embodiment 32. The device of embodiment 31, wherein the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.
Embodiment 33. The device of embodiment 31 or 32, wherein the plurality of measured parameters comprise a parameter selected from the set comprising: cardiac interbeat interval; cardiac pre-ejection period; number of skin conductance responses; and respiratory sinus arrhythmia; and mean arterial pressure.
Embodiment 34. The device of any one of embodiments 31-33, wherein the indication of positive emotional activity of the wearer comprises a score quantifying an emotion of the user selected from the set comprising: enthusiasm; sexual desire; recognition/pride; nurturant/family love; contentment; friendship love; amusement; pleasure; and gratitude.
Embodiment 35. The device of any one of embodiments 31-34, wherein the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.
Embodiment 36. The device of any one of embodiments 31-35, wherein the indication of positive emotional activity comprises a score quantifying an active emotional reward center of the user selected from the set comprising: dopamine; testosterone; serotonin; oxytocin; cannabinoids; and opioids.
Embodiment 37. The device of any one of embodiments 31-36, wherein the instructions are configured to cause the device to provide an output to the wearer indicative of the determined positive emotional activity of the wearer, wherein the output is selected from the set comprising: a displayed visualization; an audio output; and a haptic output.
Embodiment 38. The device of any one of embodiments 31-37, wherein:
the training data comprises sensor data indicating a plurality of physical or chemical parameters of the training subjects, wherein the plurality of physical or chemical parameters of the one or more training subjects are indicative of autonomic nervous system activity of the training subjects; and
the sensor data of the training data and the brain scan data of the training data were recorded simultaneously.
Embodiment 39. The device of any one of embodiments 31-38, wherein the instructions are configured to cause the system to configure, based on the training data, one or more of the plurality of sensors of the wearable device.
Embodiment 40. A method for tracking human emotion in real-time, the method performed by a wearable device comprising a plurality of sensors, one or more processors, and memory, the method comprising:
measuring, by the one or more sensors, a plurality of physical or chemical parameters of the wearer, wherein the plurality of physical or chemical parameters are indicative of autonomic nervous system activity of the wearer;
processing the plurality of measured parameters using a machine-learning-trained algorithm to generate output data, wherein the output data comprises an indication of positive emotional activity of the wearer, wherein the machine-learning-trained algorithm is trained using training data comprising brain scan data indicating brain activity of one or more training subjects, wherein the training data is labeled with information indicating positive emotional activity of the one or more training subjects at or near the time the respective sensor data was collected; and
providing an output to the wearer indicative of the determined positive emotional activity of the wearer.
Embodiment 41. The method of embodiment 40, wherein the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.
Embodiment 42. The method of embodiment 40 or 41, wherein the plurality of measured parameters comprise a parameter selected from the set comprising: cardiac interbeat interval; cardiac pre-ejection period; number of skin conductance responses; and respiratory sinus arrhythmia; and mean arterial pressure.
Embodiment 43. The method of any one of embodiments 40-42, wherein the indication of positive emotional activity of the wearer comprises a score quantifying an emotion of the user selected from the set comprising: enthusiasm; sexual desire; recognition/pride; nurturant/family love; contentment; friendship love; amusement; pleasure; and gratitude.
Embodiment 44. The method of any one of embodiments 40-43, wherein the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.
Embodiment 45. The method of any one of embodiments 40-44, wherein the indication of positive emotional activity comprises a score quantifying an active emotional reward center of the user selected from the set comprising: dopamine; testosterone; serotonin; oxytocin; cannabinoids; and opioids.
Embodiment 46. The method of any one of embodiments 40-45,comprising providing an output to the wearer indicative of the determined positive emotional activity of the wearer, wherein the output is selected from the set comprising: a displayed visualization; an audio output; and a haptic output.
Embodiment 47. The method of any one of embodiments 40-46, wherein:
the training data comprises sensor data indicating a plurality of physical or chemical parameters of the training subjects, wherein the plurality of physical or chemical parameters of the one or more training subjects are indicative of autonomic nervous system activity of the training subjects; and
the sensor data of the training data and the brain scan data of the training data were recorded simultaneously.
Embodiment 48. The method of any one of embodiments 40-47 ,comprising configuring, based on the training data, one or more of the plurality of sensors of the wearable device.
Embodiment 49. A non-transitory computer-readable storage medium storing instructions for tracking human emotion in real-time, the instructions configured to be executed by one or more processors of a wearable device comprising a plurality of sensors, to cause the wearable device to:
measure, by the one or more sensors, a plurality of physical or chemical parameters of the wearer, wherein the plurality of physical or chemical parameters are indicative of autonomic nervous system activity of the wearer;
process the plurality of measured parameters using a machine-learning-trained algorithm to generate output data, wherein the output data comprises an indication of positive emotional activity of the wearer, wherein the machine-learning-trained algorithm is trained using training data comprising brain scan data indicating brain activity of one or more training subjects, wherein the training data is labeled with information indicating positive emotional activity of the one or more training subjects at or near the time the respective sensor data was collected; and
provide an output to the wearer indicative of the determined positive emotional activity of the wearer.
Embodiment 50. The non-transitory computer-readable storage medium of embodiment 49, wherein the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.
Embodiment 51. The non-transitory computer-readable storage medium of embodiment 49 or 50, wherein the plurality of measured parameters comprise a parameter selected from the set comprising: cardiac interbeat interval; cardiac pre-ejection period; number of skin conductance responses; and respiratory sinus arrhythmia; and mean arterial pressure.
Embodiment 52. The non-transitory computer-readable storage medium of any one of embodiments 49-51, wherein the indication of positive emotional activity of the wearer comprises a score quantifying an emotion of the user selected from the set comprising: enthusiasm; sexual desire; recognition/pride; nurturant/family love; contentment; friendship love; amusement; pleasure; and gratitude.
Embodiment 53. The non-transitory computer-readable storage medium of any one of embodiments 49-52, wherein the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.
Embodiment 54. The non-transitory computer-readable storage medium of any one of embodiments 49-53, wherein the indication of positive emotional activity comprises a score quantifying an active emotional reward center of the user selected from the set comprising: dopamine; testosterone; serotonin; oxytocin; cannabinoids; and opioids.
Embodiment 55. The non-transitory computer-readable storage medium of any one of embodiments 49-54, wherein the instructions are configured to cause the device to provide an output to the wearer indicative of the determined positive emotional activity of the wearer, wherein the output is selected from the set comprising: a displayed visualization; an audio output; and a haptic output.
Embodiment 56. The non-transitory computer-readable storage medium of any one of embodiments 49-55, wherein:
the training data comprises sensor data indicating a plurality of physical or chemical parameters of the training subjects, wherein the plurality of physical or chemical parameters of the one or more training subjects are indicative of autonomic nervous system activity of the training subjects; and
the sensor data of the training data and the brain scan data of the training data were recorded simultaneously.
Embodiment 57. The non-transitory computer-readable storage medium of any one of embodiments 49-56, wherein the instructions are configured to cause the system to configure, based on the training data, one or more of the plurality of sensors of the wearable device.

### EXAMPLES

### Prophetic Example 1

In a prophetic example, peripheral signals from autonomic nervous system responses are measured using a plurality of sensors provided as part of a sensor patch. The sensors are configured to measure the following parameters indicating skin conductivity, breathing, and heart data. Specifically, the sensors are used to measure: Cardiac Interbeat Interval (IBI), ms; Cardiac Pre-Ejection Period (PEP), ms; number of valid Skin Conductance Responses (SCRs); Respiratory Sinus Arrhythmia (RSA), ms²; and Mean Arterial Pressure (MAP), mmHg.

Parameters are measured while the subject is recalling personal positive memories (e.g., while recalling said memories and/or viewing images associated with said memories). Parameters are measured for one subject across one or more runs, each run comprising four different sessions, for 18 different memories per session. Measured parameter data is labeled according to the positive human emotions that are associated with said personal positive memories.

The labeled parameter data is analyzed, manually and/or using a machine-learning training algorithm, to determine correlations between different parameter levels and various specific positive human emotions (as described above) along with intensities (quantifications) of said specific positive human emotions. An algorithm is configured (e.g., trained using ML) to process unlabeled measured parameter data and to determine, based thereon, associated specific positive human emotions (as described above) along with intensities (quantifications) of said specific positive human emotions.

The configured algorithm is then applied to unlabeled measured parameter data collected using said sensors of said sensor patch, and specific positive human emotions are thereby identified and quantified.

### Prophetic Example 2

In a prophetic example, peripheral signals from autonomic nervous system responses are measured using a plurality of sensors provided as part of a sensor patch. The sensors include the same sensors described above with reference to Prophetic Example 1, and the parameters measured are the same parameters described above with reference to Prophetic Example 1. In addition to measuring said parameters indicative of autonomic nervous system responses, fMRI brain scan data (3T rt-fMRI) is simultaneously collected.

Parameters are measured and brain scan data is measured while the subject is recalling personal positive memories (e.g., while recalling said memories and/or viewing images associated with said memories). Parameters are measured for one subject, for 60-80 total positive experiences, averaging 2-5 positive experiences per day for 21 days. Measured parameter data and brain scan data are labeled according to the positive human emotions that are associated with said personal positive memories, for example as indicated by the user when entering the user input.

The labeled parameter data and brain scan data is analyzed, manually and/or using a machine-learning algorithm, to determine correlations between different parameter levels, different brain scan data characteristics, and various specific positive human emotions (as described above) along with intensities (quantifications) of said specific positive human emotions. An algorithm is configured (e.g., trained using ML) to process unlabeled measured parameter data collected from said patch sensors and to determine, based thereon, associated specific positive human emotions (as described above) along with intensities (quantifications) of said specific positive human emotions. Said algorithm is trained based at least in part on said collected brain scan data, but is trained to make determinations based on patch sensor data alone (without reference to corresponding brain scan data).

The configured algorithm is then applied to unlabeled measured parameter data collected using said sensors of said sensor patch, and specific positive human emotions are thereby identified and quantified.

### Prophetic Example 3

In a prophetic example, peripheral signals from autonomic nervous system responses are measured using a plurality of sensors provided as part of a sensor patch. The sensors include the same sensors described above with reference to Prophetic Example 1, and the parameters measured are the same parameters described above with reference to Prophetic Example 1.

Parameters are measured and recorded from time windows during which the subject indicates a positive experience is ongoing or was recently completed. For example, the subject uses an input device, such as an input device having a graphical user interface (e.g., a smart-phone application) to indicate that a positive experience is ongoing or was recently completed. The input by the user causes parameters measured by the sensor patch to be recorded and/or analyzed. For example, the input by the user causes the sensor patch to start measuring. In another example, the input by the user causes the system to start recording measurements from the sensor patch. In another example, the input by the user causes the system to durably record and/or to analyze parameters that were recorded and temporarily stored by the system at a time indicated by the user's input, to be associated with the positive experience. Parameters are measured for one subject, across one or more runs, each run comprising four different sessions, for 18 different memories per session. Measured parameter data is labeled according to the positive human emotions that are associated with said personal positive memories.

The labeled parameter data is analyzed, manually and/or using a machine-learning training algorithm, to determine correlations between different parameter levels and various specific positive human emotions (as described above) along with intensities (quantifications) of said specific positive human emotions. An algorithm is configured (e.g., trained using ML) to process unlabeled measured parameter data collected from said patch sensors and to determine, based thereon, associated specific positive human emotions (as described above) along with intensities (quantifications) of said specific positive human emotions.

The configured algorithm is then applied to unlabeled measured parameter data collected using said sensors of said sensor patch, and specific positive human emotions are thereby identified and quantified.

### Prophetic Example 4

In a prophetic example, peripheral signals from autonomic nervous system responses are measured using a plurality of sensors provided as part of a sensor patch. The sensors include the same sensors described above with reference to Prophetic Example 1, and the parameters measured are the same parameters described above with reference to Prophetic Example 1.

Parameters are measured and recorded from time windows during which the subject indicates a positive experience is ongoing or was recently completed. For example, the subject uses an input device, such as an input device having a graphical user interface (e.g., a smart-phone application) to indicate that a positive experience is ongoing or was recently completed, as described above with reference to Prophetic Example 3. Parameters are measured for 20-24 subjects (having a 50:50 male: female split, 18-35 years old, with no neurological or psychiatric disorders). For each subject, data for 84 total positive experiences are recorded over 6 weeks. Measured parameter data is labeled to indicate the subject and the positive human emotions that are associated with said personal positive memories, for example as indicated by the user when entering the user input.

In addition to collecting sensor data during real-life experiences, parameters are measured using the patch sensors and brain scan data is collected using 3T fMRI during one or more brain scan sessions. During a session, data for each subject is measured while the subject recalls a peak memory (a “best of life” memory). The data pattern between emotional experience in real-time and during memory recall in the MRI is highly similar. Measured parameter data and brain scan data from said sessions are labeled to indicate the subject and the positive human emotions that are associated with said peak memories, for example as indicated by the subject.

The labeled parameter data and brain scan data is analyzed, manually and/or using a machine-learning algorithm, to determine correlations between different parameter levels, different brain scan data characteristics, and various specific positive human emotions (as described above) along with intensities (quantifications) of said specific positive human emotions. An algorithm is configured (e.g., trained using ML) to process unlabeled measured parameter data collected from said patch sensors and to determine, based thereon, associated specific positive human emotions (as described above) along with intensities (quantifications) of said specific positive human emotions. Said algorithm is trained based at least in part on said collected brain scan data, but is trained to make determinations based on patch sensor data alone (without reference to corresponding brain scan data).

The configured algorithm is then applied to unlabeled measured parameter data collected using said sensors of said sensor patch, and specific positive human emotions are thereby identified and quantified.

Follow-up scans are performed after 3 months and 6 months. Structural changes in cortical thickness and rGMV are in line with emotional ratings and overall happiness ratings.

### Prophetic Example 5

In a prophetic example, a stepwise manner of gathering wearable sensor training data is performed to address the effect of electrophysiological signals, such as those produced by muscle contractions, on autonomic nervous system data.

First, the subject is placed in a 7T MRI, wearing one or more research-grade wearables and one or more consumer wearables including the necessary sensors. Upon presentation of positive memory triggers (e.g., photos, videos, music, text, audio) the real-time changes measured in the emotion centers in the brain from the fMRI are harmonized with outputs from the research-grade wearable(s) and the consumer wearable. The research-grade wearable serves as a control with a higher sensitivity as compared to the consumer wearable. This data harmonization is enhanced using machine-learning (e.g., generative AI). A refined consumer wearable is produced that identifies and quantifies the nine emotions.

After the MRI scan, the subject completes a post-hoc emotion assessment to rate his/her subjective emotions. The post-hoc assessment aligns subjective feelings to objective brain activity measures and objective wearable measures for further calibration and/or optimization of the wearable device.

After the MRI scan, the algorithm training is then repeated outside the MRI with the subject standing still in front of a screen projecting the same positive memory triggers. Any noise that is added when there is basic muscle contraction in an upright, standing position is measured. The post-hoc subjective emotion rating by the subject is repeated. Any noise from standing is deducted from the measured wearable data.

The algorithm training is then repeated on a treadmill with the subject walking while the same positive memory triggers are projected on a screen. Any noise that is added when there is active muscle contraction while walking is measured. The post-hoc subjective emotion rating is repeated. Any noise from walking is then deducted from the measured wearable data.

A continuous machine-learning training process of the algorithm is then performed, as follows. Whenever the wearable sensor(s) detects a signal above a noise baseline, the wearable device prompts the subject to confirm, correct, or decline a measured emotion determined based on the detected signal. Over time, the subject trains the wearable device to recognize sensor data patterns above the noise baseline. For example, the wearable device detects an increase in arousal (demonstrated by skin conductivity change and/or heart rate variability, or HRV, change), and in response, using a graphical user interface of an input device, prompts the following question (or similar) to the subject: “Has anything positive happened?”. When the subject confirms via the input device that yes, something positive happened, the wearable device then prompts the subject to indicate whether the positive event was accompanied by, for example, feelings of excitement, sexual desire, amusement, or pleasure. Upon selection of one or more of the emotions, the underlying sensor data pattern is refined and prioritized for future uses. When the subject does not confirm that something positive has happened because the arousal is ultimately triggered by a negative experience (e.g., anxiety, panic, stress, etc.) the ML-trained algorithm uses this information to deprioritize the underlying sensor data pattern. In some examples, the post-hoc emotion assessment includes the wearable device prompting the subject to quantify the emotion intensity on a scale, such as a scale from 0-8. Over time (e.g., after 2-3 weeks), the wearable device is fine-tuned to be able to efficiently detect and quantify the nine specific emotions.

### Conclusion

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

For any numerical ranges disclosed in the text and figures, the numerical ranges disclosed inherently support any range or value within the disclosed numerical ranges, including the endpoints, even though a precise range limitation is not stated verbatim in the specification, because this disclosure can be practiced throughout the disclosed numerical ranges.

The above description is presented to enable a person skilled in the art to make and use the disclosure, and it is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown but is to be accorded the widest scope consistent with the principles and features disclosed herein.

Finally, the entire disclosure of the patents and publications referred in this application are hereby incorporated herein by reference.

1. A system for tracking human emotion in real-time, the system comprising:
a wearable device comprising a plurality of sensors;
one or more processors; and
memory storing instructions configured to cause the system to:
measure, by the one or more sensors, a plurality of physical or chemical parameters of the wearer, wherein the plurality of physical or chemical parameters are indicative of autonomic nervous system activity of the wearer; and
process the plurality of measured parameters using a machine-learning-trained algorithm to generate output data, wherein the output data comprises an indication of positive emotional activity of the wearer.

2. The system of claim 1, wherein the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.

3. The system of claim 1, wherein the plurality of measured parameters comprise a parameter selected from the set comprising: cardiac interbeat interval; cardiac pre-ejection period; number of skin conductance responses; and
respiratory sinus arrhythmia; and mean arterial pressure.

4. The system of claim 1, wherein the indication of positive emotional activity of the wearer comprises a score quantifying an emotion of the user selected from the set comprising: enthusiasm; sexual desire; recognition/pride;
nurturant/family love; contentment; friendship love; amusement; pleasure; and gratitude.

5. The system of claim 1, wherein the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.

6. The system of claim 1, wherein the indication of positive emotional activity comprises a score quantifying an active emotional reward center of the user selected from the set comprising: dopamine; testosterone; serotonin;
oxytocin; cannabinoids; and opioids.

7. The system of claim 1, wherein the instructions are configured to cause the system to provide an output to the wearer indicative of the determined positive emotional activity of the wearer, wherein the output is selected from the set comprising: a displayed visualization; an audio output; and a haptic output.

8. The system of claim 1, wherein the instructions are configured to cause the system to:
receive training data comprising brain scan data indicating brain activity of one or more training subjects, wherein the training data is labeled with information indicating positive emotional activity of the one or more training subjects at or near the time the respective sensor data was collected; and
train, using the training data, the machine-learning-trained algorithm.

9. The system of claim 8, wherein:
the training data comprises sensor data indicating a plurality of physical or chemical parameters of the training subjects, wherein the plurality of physical or chemical parameters of the one or more training subjects are indicative of autonomic nervous system activity of the training subjects; and
the sensor data of the training data and the brain scan data of the training data were recorded simultaneously.

10. The system of claim 1, wherein the instructions are configured to cause the system to:
receive training data comprising brain scan data indicating brain activity of one or more training subjects, wherein the training data is labeled with information indicating positive emotional activity of the one or more training subjects at or near the time the respective sensor data was collected; and
configure, based on the training data, one or more of the plurality of sensors of the wearable device.

11. A method for tracking human emotion in real-time, the method performed at a system comprising a wearable device comprising a plurality of sensors, one or more processors, and memory, the method comprising:
measuring, by the one or more sensors, a plurality of physical or chemical parameters of the wearer, wherein the plurality of physical or chemical parameters are indicative of autonomic nervous system activity of the wearer; and
processing the plurality of measured parameters using a machine-learning-trained algorithm to generate output data, wherein the output data comprises an indication of positive emotional activity of the wearer.

12. (canceled)

13. A wearable device for tracking human emotion in real-time, the wearable device comprising:
a plurality of sensors;
one or more processors; and
memory storing instructions configured to cause the device to:
measure, by the one or more sensors, a plurality of physical or chemical parameters of the wearer, wherein the plurality of physical or chemical parameters are indicative of autonomic nervous system activity of the wearer;
process the plurality of measured parameters using a machine-learning-trained algorithm to generate output data, wherein the output data comprises an indication of positive emotional activity of the wearer, wherein the machine-learning-trained algorithm is trained using training data comprising brain scan data indicating brain activity of one or more training subjects, wherein the training data is labeled with information indicating positive emotional activity of the one or more training subjects at or near the time the respective sensor data was collected; and
provide an output to the wearer indicative of the determined positive emotional activity of the wearer.

14. The device of claim 13, wherein the one or more sensors comprise a sensor selected from the set comprising: an electrical activity sensor; a photoplethysmography sensor; a non-contact system configured for measuring arterial pulse; a sensors based on mechanical activity; a forcecardiography sensor; a seismocardiography sensor; an ECG sensor; an impedance cardiography sensor; a sensor detecting galvanic skin response; a pressure-based mean arterial pulse sensor; and an ultrasound sensor.

15. The device of claim 13, wherein the plurality of measured parameters comprise a parameter selected from the set comprising: cardiac interbeat interval;
cardiac pre-ejection period; number of skin conductance responses; and respiratory sinus arrhythmia; and mean arterial pressure.

16. The device of claim 13, wherein the indication of positive emotional activity of the wearer comprises a score quantifying an emotion of the user selected from the set comprising: enthusiasm; sexual desire; recognition/pride;
nurturant/family love; contentment; friendship love; amusement; pleasure; and gratitude.

17. The device of claim 13, wherein the indication of positive emotional activity of the wearer comprises a score quantifying respective emotions of the user for all emotions in the set comprising: enthusiasm; sexual desire; recognition/pride; nurturant/family love; contentment; friendship love; amusement; pleasure; and gratitude.

18. The device of claim 13, wherein the indication of positive emotional activity comprises a score quantifying an active emotional reward center of the user selected from the set comprising: dopamine; testosterone; serotonin; oxytocin; cannabinoids; and opioids.

19. The device of claim 13, wherein the instructions are configured to cause the device to provide an output to the wearer indicative of the determined positive emotional activity of the wearer, wherein the output is selected from the set comprising: a displayed visualization; an audio output; and a haptic output.

20. The device of claim 13, wherein:
the training data comprises sensor data indicating a plurality of physical or chemical parameters of the training subjects, wherein the plurality of physical or chemical parameters of the one or more training subjects are indicative of autonomic nervous system activity of the training subjects; and
the sensor data of the training data and the brain scan data of the training data were recorded simultaneously.

21. The device of claim 13, wherein the instructions are configured to cause the system to configure, based on the training data, one or more of the plurality of sensors of the wearable device.

22. (canceled)

23. (canceled)
